(19) United States
(12) Patent Application Publication
(10) Pub. No.: US 2011/0160840 A1
(43) Pub. Date: Jun. 30, 2011

(54) Repetitive Cell Bifurcation / Side Branch Ostia Support Stent

(75) Inventors: Michael P. Meyer, Richfield, MN (US); Alison Julson, Ramsey, MN (US); Timothy J. Mickley, Corcoran, MN (US)

(73) Assignee: BOSTON SCIENTIFIC SCIMED, INC., Maple Grove, MN (US)

(21) Appl. No.: 12/964,175

(22) Filed: Dec. 9, 2010

Related U.S. Application Data
(60) Provisional application No. 61/291,526, filed on Dec. 31, 2009.

(51) Int. Cl. A61F 2/82

(52) U.S. Cl. 623/1.15

(57) ABSTRACT

A stent comprises at least one closed pathway that defines a single cell. In some embodiments, the closed pathway includes a plurality of petals constructed and arranged to extend away from the tubular wall of the stent. In one embodiment, some of the petals of the closed pathway overlap other of the petals of the closed pathway when the stent is in a reduced state. In at least one embodiment, the stent comprises a plurality of closed pathways. In one embodiment, the closed pathways are arranged in bands that extend either along the length of the stent or about the circumference of the stent.

### CROSS-REFERENCE TO RELATED APPLICATIONS

This application is a non-provisional of application No. 61/291,526, filed Dec. 31, 2009.

### STATEMENT REGARDING FEDERALLY SPONSORED RESEARCH

Not Applicable

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to a stent comprising at least one closed pathway defining a single cell. In some embodiments, the closed pathway includes a plurality of petals constructed and arranged to extend away from the tubular wall of the stent. In one embodiment, some of the petals of the closed pathway overlap other of the petals of the closed pathway when the stent is in a reduced diameter or reduced state. In at least one embodiment, the stent comprises a plurality of closed pathways. In one embodiment, the closed pathways are arranged in bands that extend either along the length of the stent, about the circumference of the stent, or both along the length of the stent and about the length of the stent and about the circumference of the stent.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIGS. 1-4 are flat views of closed pathway structures. FIG. 3B shows the closed pathway structure of FIG. 3A oriented at a 90 degree angle.

FIGS. 5-9B are flat views of the closed pathway structures of FIGS. 1-4 interconnected to form a pattern.

FIGS. 10-11 are flat views of closed pathway structures.

FIGS. 12-14 are flat views of the closed pathway structures of FIGS. 10-11 interconnected to form a pattern.

FIG. 15 is a schematic flat view of interconnected closed pathways extending along the length of a stent.

FIG. 16 is a schematic flat view of interconnected closed pathways forming a circumferential section of a stent.

FIG. 17 is a flat view of interconnected closed pathways with radiopaque markers.

FIG. 18 is a flat view of the pattern of FIG. 5 in a reduced state.

FIG. 19 is a flat view of the pattern of FIG. 5 where one closed pathway has been expanded to form a side branch.

FIGS. 20-24 show a closed pathway of the stent being expanded to form a side branch with a guide wire extending through the closed pathway into a side branch vessel. FIG. 22a shows the pattern of FIG. 5 with a guide wire extending through one of the plurality of closed pathways. FIG. 22b shows the pattern of FIG. 14 with a guide wire extending wire extending through one of the plurality of closed pathways.

FIGS. 25-26 show a closed pathway of the stent being expanded to form a side branch extending into a side branch vessel by a second balloon catheter.

FIG. 27 is a schematic view of a stent comprising a plurality of interconnected closed pathways wherein two of the close pathways are expanded to form side branches deployed into two ostiums of a body lumen.

FIG. 28 is a schematic view of FIG. 17 illustrating the use of the markers to position a closed pathway at the ostium.

FIGS. 29A-29G are schematic illustrations of a crimping procedure.

FIG. 30A shows a hinge made by reducing the width of a member.

FIG. 30B shows a hinge made by reducing the thickness of a member.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

As used in this application, the stent is comprised of a plurality of members 20. Members 20 include struts 22, turns 24, petals 26, hinges 28, connectors 38, and turns 48. Each member 20 has a width, a length, and a thickness where the width is perpendicular to the length of the member 20 and the thickness of the member 20 is measured radially from the outside surface of the member 20 to the inside surface of a member 20.

Although the figures in this application show the stent 10 in a flat, plan view, in use, the stent 10 has a tubular body. Although, the wall of the tubular body has curvature, terms, for example, “straight” or “curvilinear,” used herein to describe the shape of the members 20, forming the stent, do not include the curvature of the tubular body.

In at least one embodiment, a stent 10 comprises at least one closed pathway pathway 12. As shown in the figures, each closed pathway extends partway about the circumference of the stent and partway along the longitudinal length of the stent. Each closed pathway 12 is formed by a plurality of members 20 that entirely define a cell 14. Thus, no members 20 other than those of the closed pathway 12 define the cell 14. As used in this application, a cell 14 or an opening is an area completely bounded by a plurality of members 20 and does not include openings within a single member 20. An opening completely bounded by a plurality of members 20 include openings indicated by reference numerals 35, 36, 40, 43, and 44. Similarly, a side branch opening 14a, discussed below, is an area completely bounded by a plurality of members 20 forming a side branch.

In some embodiments, a stent 10 comprises a plurality of closed pathways 12. In other embodiments, the stent 10 comprises at least one closed pathway 12 and additional stent structure 32 engaged to the at least one closed pathway 12. In at least one embodiment, the additional stent structure 32 comprises a plurality of interconnected members 20. In some embodiments, the interconnected members 20 include struts 22 that are interconnected by proximal turns and distal turns.

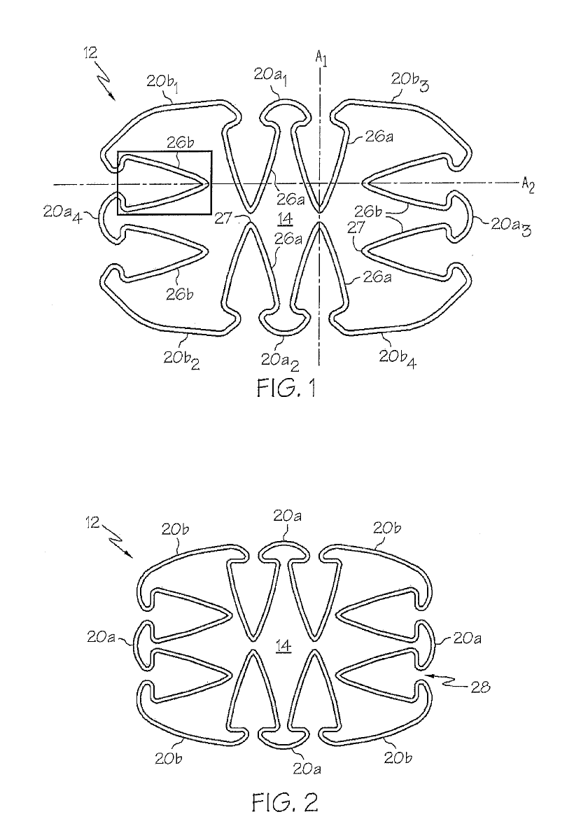

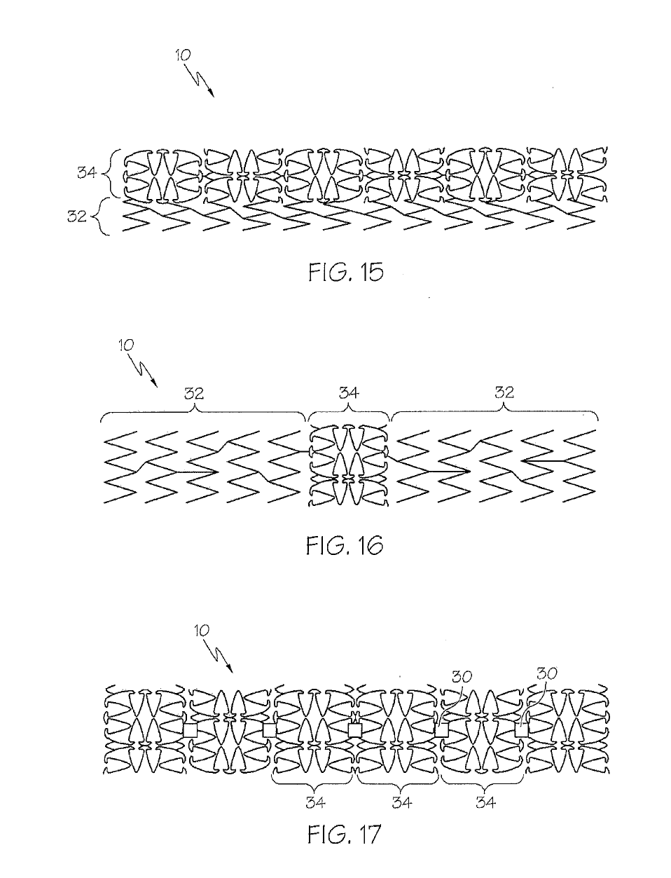

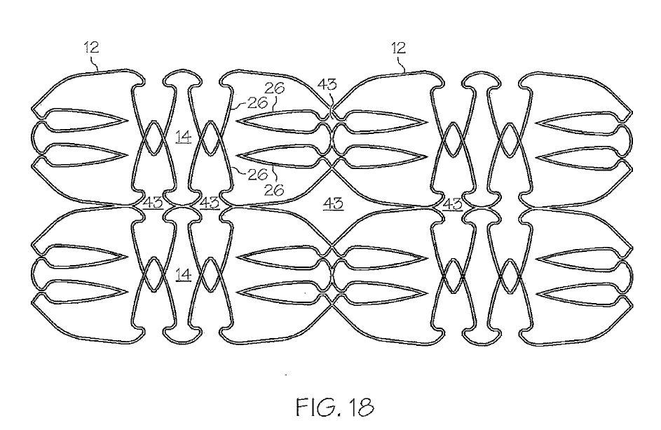

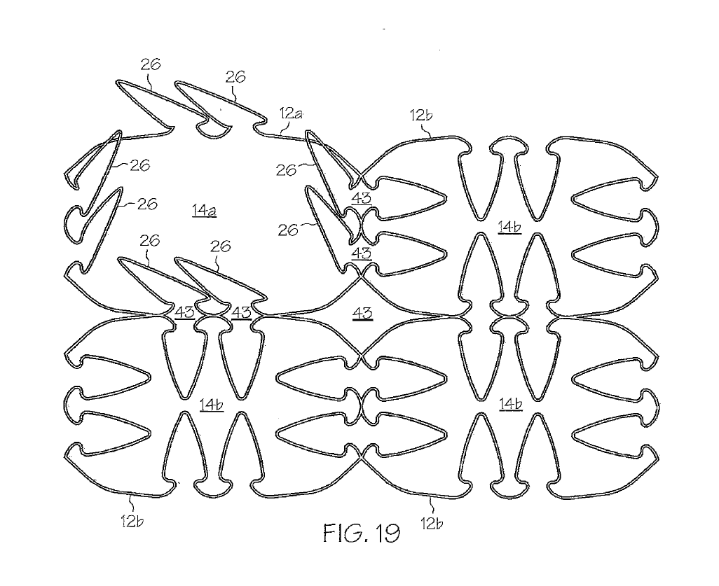

FIGS. 1-4 and 9A-10 show different embodiments of closed pathways 12 formed by a plurality of members 20 that define a cell 14. For simplicity, only the structure of the closed pathway 12 is shown. FIGS. 15 and 16 show non-limiting examples of stent structure 32 engaged to at least one closed pathway 12 to form a stent 10. In at least one embodiment, the closed pathway 12 is a side branch when at least one portion of the closed pathway 12 extends away from the tubular wall of the stent. If the closed pathway 12 is a side branch, then the cell 14 is a side branch opening 14a, as shown for example by closed pathway 12a in FIG. 19.

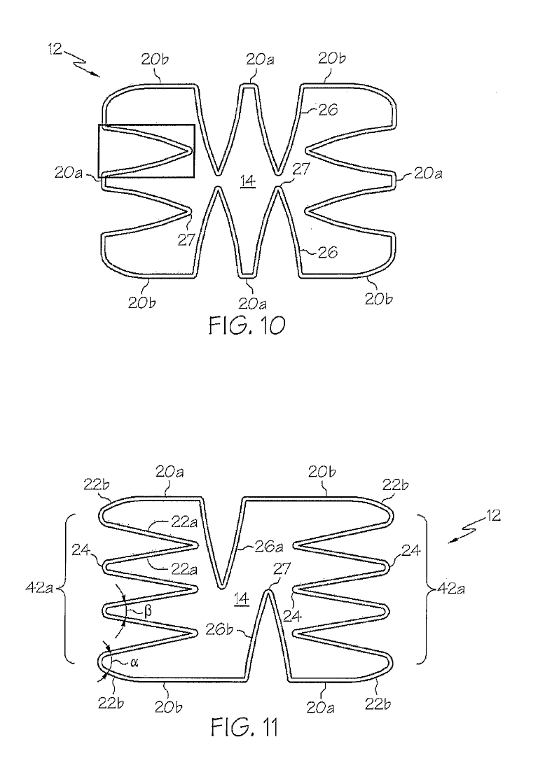

In at least one embodiment, the closed pathway 12 includes at least one petal 26 that extends into the interior of cell 14, as shown for example in FIGS. 1-4 and 10-11. Non-limiting examples of structures for a petal 26 are shown in FIGS. 1 and 10 by a box surrounding a petal 26. As used in this application, a petal 26 is a structure that is constructed and designed to extend away from the tubular body of the stent to form a portion of a side branch. As is in known in the art, when coverage of a side vessel branching off of a main vessel is desired, a side branch can be extended away from the tubular body of the stent into the side vessel.

In some embodiments, the petals 26 have a hinge 28, as shown for example in FIGS. 1-4. Without being bound by theory, the hinge 28 provides a focal point for bending to occur when the closed pathway 12 is expanded to form a side branch. Thus, as used in this application a hinge is a focal point for bending. Non-limiting examples of hinges are discussed below.

In at least one embodiment, the hinge 28 allows for optimal extension of at least a portion of a member 20 into the side branch. As shown for example in FIG. 1, each petal 26 is made of straight segments and curved segments. As shown the curved segments are at the first/base end region of the petal. Without being bound by theory, when a force is applied to a petal 26 for example by a balloon, the straight segments remain straight and the curved segments will bend, as shown in FIG. 19. Thus, in this embodiment, the hinge 28 is a curved segment. In some embodiments, the straight segments of the petal act as a level and force the bending or torsion of the curved segments at the first/base end region of the petal. Without being bound by theory, it takes less force to bend the curved segments than the straight beams. In some embodiments, the curved segments have a smaller width than the straight segments.

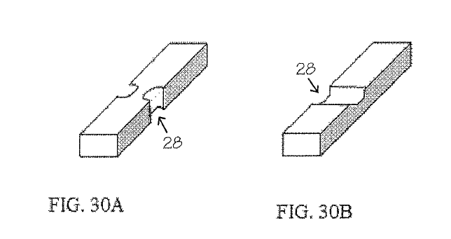

In other embodiments, the petals 26 do not have a hinge in the form of a curved segment, as shown for example in FIGS. 10-11. In this embodiment, strut wall thickness is optimized so that bending can occur symmetrically similar to that shown in FIG. 19 with a hinge 28 in the form of a curved segment. In some embodiments, a hinge 28 is made by reducing the width of a portion of the member 20. Without being bound by theory, for a given thickness of a member, the thinner the width of the member, the easier it is for the member to bend or twist. Thus, this provides a means to focalize where a member 20, such as a petal 26, will bend. In other embodiments, a hinge 28 is made by reducing the thickness of a portion of the member 20. In at least one embodiment, the member 20 has hinges 28 which are symmetrically placed so that the member 20 opens in a symmetrical manner as discussed above. FIGS. 30A and 30B respectively show a non-limiting example of a hinge 28 made by reducing the width of a member and a non-limiting example of a hinge 28 made by reducing the thickness of the member. In some embodiments, material is removed from a member by a laser to form a hinge.

The closed pathways 12 shown in FIGS. 1-4 and 10 each have eight (8) petals petals 26. It is within the scope of the invention for a closed pathway 12 to have one, two, three, four, five, six, seven, eight, or more petals 26. As shown in the figures, each petal 26 in a closed pathway 12 has the same petal shape and the same size. It is within the scope of the invention for the petals 26 to have any shape defining an opening. In at least one embodiment, the petal 26 has a first/base end region and a second/tip end region 27 with the second/tip end region 27 of the petal 26 extending away from the tubular body when the petal forms a part of a side branch that is in an expanded state.

In some embodiments, the petals 26 define an arrow-shaped opening, as shown for example in FIGS. 1-3. In the embodiment, the portion of the opening defined by the second/tip end region 27 of the petal is less than the portion of the opening defined by first/base end region of the petal. In other embodiments, the petals define a triangular shaped opening, as shown for example in FIGS. 1-3 and 10-11. Although the petals shown in the figures define an arrow shaped opening or a triangular shaped opening, it is within the scope of the invention for the petals 26 to have any shape and to define any shaped opening. For example the opening defined by the petal can be triangular shaped, arrow shaped, round shaped, rectangular shaped, multi-lobed, and any combination thereof.

In at least one embodiment, each petal 26 of a closed pathway is bisected by an axis A. In FIG. 1, some of the petals 26 are bisected by axis A₁ and some of the petals 26 are bisected by axis A₂. In some embodiments, axes bisecting petals of a closed pathway are perpendicular to one another. For example, as shown in FIG. 1, axis A₁ is perpendicular to axis A₂. Note that the closed pathway 12 shown in FIG. 1 has additional axes bisecting at least one petal 26 that are not labeled which are parallel to the labeled axes A₁, A₂. Thus, it is within the scope of the invention for a closed pathway 12 to have a plurality of first axes A₁ and a plurality of second axes A₂.

In some embodiments, at least one axis bisects some of the petals 26 of the closed pathway 12 and intersects other of the petals 26 of the closed pathway 12. This is shown for example in FIG. 1 where axis A₁ bisects petals 26b and intersects petals 26a. In at least one embodiment, the plurality of petals of a closed pathway comprise a plurality of first petals and a plurality of second petals, each first petal being bisected by a first axis and each second petal being bisected by a second axis where the first axis is perpendicular to the second axis and wherein one of the first axes intersects at least one of the second petals. This least one of the second petals. This is shown for example in FIG. 1.

In some embodiments, the petals 26 are engaged to form pairs of petals, as shown for example in FIGS. 1-4 and 10. In FIGS. 1-3 the petals 26 in a pair are engaged by member 20a. In some embodiments, the shape of member 20a is curvilinear, as shown for example in FIG. 1. In other embodiments, the shape of member 20a is straight/linear, as shown for example in FIG. 10. Non-limiting examples of different curvilinear shapes for member 20a are shown in FIGS. 1-3. In some embodiments, members 20a of a closed pathway 12 are positioned opposite one another. Thus, as shown for example in FIG. 1, member 20a₁ is positioned opposite member 20a₂ and member 20a₃ is positioned opposite member 20a₄.

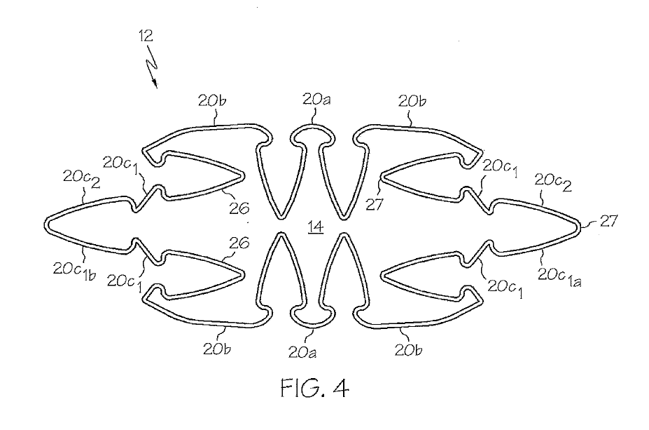

In still other embodiments, the petals 26 in a pair are engaged by a member 20c comprising end portions 20c₁ that each have a first shape and a middle portion 20c₂ that has a second shape, as shown for example in FIG. 4. In some embodiments, the shape of the middle portion 20c₂ is the same shape as the petals 26, as shown in FIG. 4. In other embodiments, members 20c of a closed pathway 12 are positioned opposite one another. Thus, as shown for example in FIG. 4, member 20c₁ₐ is positioned opposite member 20c_{1b}.

Pairs of petals 26 are aligned and positioned opposite another pair of petals 26. Thus, one pair of petals 26 is aligned in a first direction and the other pair of petals is aligned in a second direction opposite the first direction. As used in this application petals 26 aligned in opposite directions are positioned 180 degrees from one another. Thus, as shown in FIG. 1, the second/tip end region 27 of petals 26 bisected by axis A₁ are pointed towards one another and the second/tip end region 27 of petals 26 bisected by axis A₂ are pointed towards one another.

In at least one embodiment, member 20b engages adjacent pairs of petals together. In some embodiments, member 20b is curvilinear. Non-limiting examples of the different curvilinear shapes that member 20b can have are shown in FIGS. 1-3 and 10. As can be seen from the figures, the shape of member 20b is different from the shape of member 20a and the length of member 20b is greater than the length of member 20a. However, it is within the scope of the invention for members 20a,b to have any length and shape. In some embodiments, members 20b of a closed pathway 12 are positioned opposite one another. Thus, as shown for example in FIG. 1, member 20b₁ is positioned opposite member 20b₂ and opposite member 20b₂ and member 20b₃ is positioned opposite member 20b₄.

In at least one embodiment, the closed pathway 12 comprises a plurality of members 20 which include a plurality of first members 20b, a plurality of second members 20a, and a plurality of petals 26, as shown for example, in FIGS. 1-4 and 10. In some embodiments, each first member 20b has a first shape, each second member 20a has a second shape and each petal 26 has a petal shape where the first shape is different from the second shape and the petal shape and the second shape is different from the petal shape. In one embodiment, the plurality of members 20 of the closed pathway is arranged as follows: first member 20b-petal 26-second member 20a-petal 26-first member 20b-petal 26-second member 20a-petal 26-first member 20b-petal 26-second member 20a-petal 26-first member 20b-petal 26-second member 20a-petal 26. This arrangement of members 20 is shown for example in FIGS. 1-3 and 10.

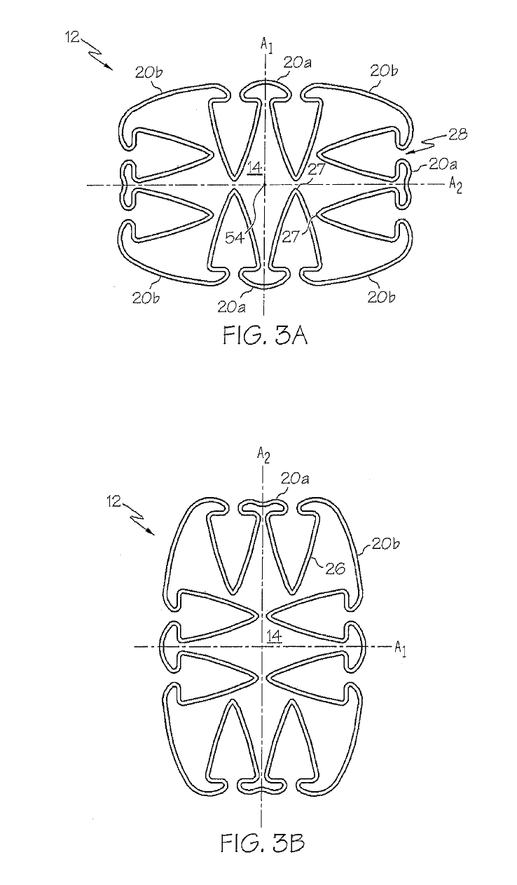

Note that the structure of the closed pathway 12 and the cell 14 defined thereby, depends at least on the arrangement of the member, the number of members, and the shape of each member. In at least one embodiment, the structure of the closed pathway 12 has a circumferential extent and a longitudinal extent. In some embodiments, the circumferential extent is less than the longitudinal extent, as shown for example in FIG. 3A. In other embodiments, the circumferential extent is greater than the longitudinal extent, as shown for example in FIG. 3B.

In some embodiments, the plurality of members 20 of the closed pathway 12 further includes a plurality of third members 20c with each third member 20c having a third structure different from each of the first, second, and petal shapes. As shown for example in FIG. 4, the closed pathway 12 comprises a plurality of first members 20b, a plurality of second members 20a, a plurality of third members 20c, and a plurality of petals 26. In one embodiment, the plurality of members 20 of the closed pathway 12 is arranged as follows: first member 20b-petal 26-second member 20a-petal 26-first member 20b-petal 26-third member 20c-petal 26-first member 20b-petal 26-second member 20a-petal 26-first member 20b-petal 26-third member 20c-petal 26. This arrangement of members 20 is shown for example in FIG. 4.

In at least one embodiment, the closed pathway 12 extends about a center point 54 at an intersection of a first axis bisecting the cell 14 in a first direction and a second axis bisecting the cell 14 in a second direction which is 90 degrees to the first direction. In some embodiments the closed pathway 12 is symmetrical about two axes. For example, as shown in FIG. 3, the closed pathway 12 is symmetrical about axis A₁ and about axis A₂. In this embodiment, the cell 14 defined by the closed pathway 12 is also symmetrical about the two axes A₁ and A₂.

In other embodiments, the closed pathway 12 is asymmetrical. For example the closed pathway 12 in FIG. 11 is not symmetrical about any axes (asymmetrical). In this embodiment, the cell 14 defined by the closed pathway 12 is also asymmetrical.

It is within the scope of the invention for the closed pathway 12 to be oriented as shown in the figures or at any angle relative to the longitudinal axis of the stent. For example, the closed pathways in FIGS. 3A and 3B have the same structure but different orientations with the closed pathway 12 in FIG. 3A oriented with axis A₂ parallel to the longitudinal axis of the stent and the closed pathway 12 in FIG. 3B oriented so that axis A₂ is perpendicular to the longitudinal axis of the stent.

FIG. 11 shows a closed pathway 12 that has two (2) petals 26 that are directed in opposite directions. Because the petals 26 are offset from one another, they are not aligned on a common axis. Each petal 26 is engaged to two rows of interconnected struts 22 by a member 20. In this embodiment, member 20 is straight/linear. The interconnected struts 22 comprise long struts 22a and short struts 22b. As shown in FIG. 11, the interconnected struts 22 are serpentine because the turns 24 are rounded. However, it is within the scope of the invention for the interconnected struts 22 to be zig-zag with pointed turns. Each short strut 22b is engaged to a member 20 and to a long strut 22a. In some embodiments, the turn α engaging a short strut 22b to a long strut 22a has a greater angle than the turn β engaging two long struts 22a, as shown in FIG. 11. In other embodiments, the turn a engaging a short strut 22b to a long strut 22a is the same angle as the turn β engaging two long struts 22a (not shown).

In at least one embodiment, the closed pathway 12 includes a plurality of members 20, which include a plurality of struts 22 and a plurality of petals 26, as shown for example in FIG. 11. In some embodiments, the plurality of struts 22 includes a plurality of plurality of first struts 22a each having a first length and a plurality of second struts 22b each having a second length less than the first length. In one embodiment, the plurality of struts 22 is interconnected about a portion of the circumference of the stent to form a first band 42a and a second band 42b. In some embodiments, the plurality of members 20 includes a plurality of first members 20a having a first length and a plurality of second members 20b having a second length greater than the first length. In one embodiment, a first member 20a is engaged to the first band 42a and to a first petal 26a and a first member 20a is engaged to a second band 42b and to a second petal 26b and a second member 20b is engaged to the first band 42a and to the first petal 26a and a second member 20b is engaged to the second band 42b and to the second petal 26b. In some embodiments, the first and second members 20a,b are parallel to the longitudinal axis of the stent. In at least one embodiment, the first and second petals 26a,b, are longitudinally offset from one another.

In at least one embodiment, the closed pathway 12 has the following arrangement of members 20: first member 20a-petal 26-second member 20b-circumferential band of struts 42b-first member 20a-petal-second member 20b-circumferential band of struts 42a. This arrangement of members 20 is shown for example in FIG. 11.

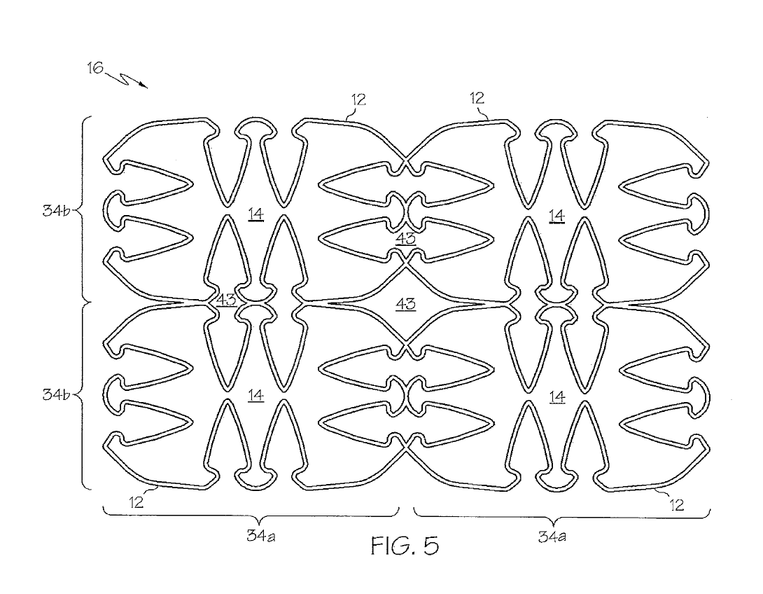

FIGS. 5-9B and 12-14 show non-limiting examples of a plurality of closed pathways 12 interconnected to form at least a portion of a stent pattern 16. In at least one embodiment, the stent comprises a plurality of closed pathways 12 where each closed pathway 12 comprises a plurality of members 20 arranged in the same manner, for example each closed pathway comprises six members 20 arranged 1-2-3-4-5-6. In some embodiments, each of the plurality of closed pathways 12 in a pattern has the same orientation relative to the longitudinal axis of the stent. In other embodiments, some of the plurality of closed pathways 12 have a different orientation relative to the longitudinal axis of the stent than other of the closed pathways 12.

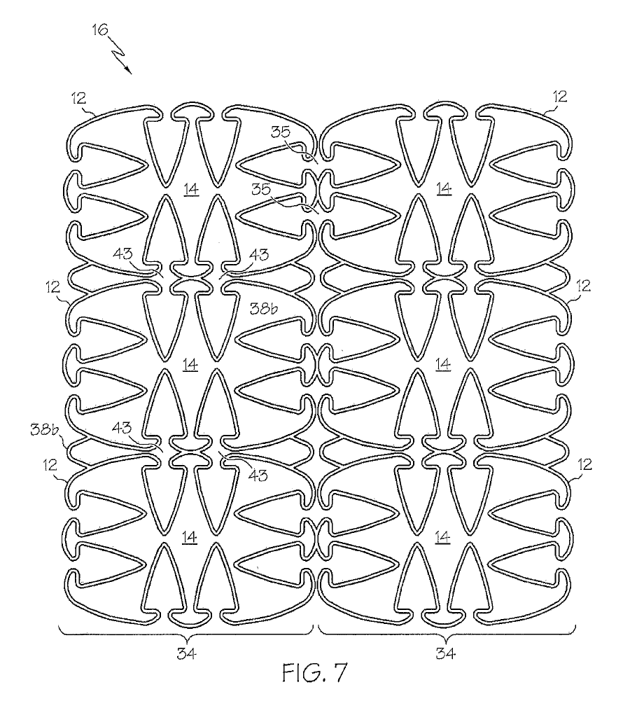

It is within the scope of the invention for adjacent closed pathways 12 to be interconnected at one, two, three, four, five, six or more places. For example, in FIG. 5, each closed pathway 12 is interconnected with an adjacent closed pathway at least three locations on each side of the closed pathway 12. In FIG. 7, each closed pathway 12 is interconnected with an adjacent closed pathway 12 at only one location, excluding connectors connectors 38.

In some embodiments, closed pathways 12 are interconnected by abutting one another, as shown for example in FIGS. 5, 7 and 12-14. In other embodiments, adjacent closed pathways 12 are interconnected by having elements in common. In still other embodiments, closed pathways 12 are interconnected by connectors 38. Thus it is within the scope of the invention for the closed pathway 12 to be interconnected by abutting, having elements in common, connectors, and any combination thereof.

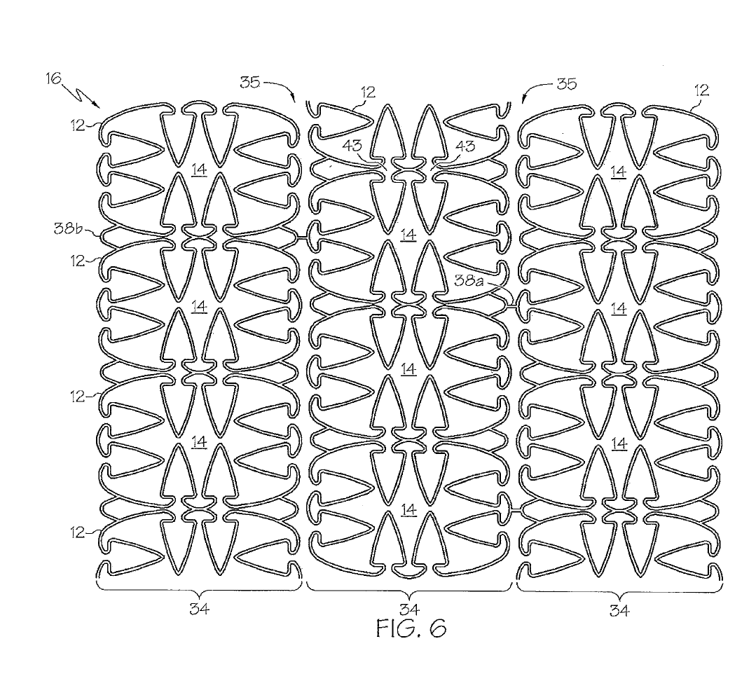

In FIG. 5, adjacent closed pathways 12 share the entire length of members 20a and a portion of the length of members 20b. In FIG. 7, adjacent closed pathways 12 share a portion of members 20a. In other embodiments, adjacent closed pathways are interconnected by connectors 38b, as shown for example in FIGS. 6 and 7. In some embodiments, connector 38b adds radial support. The shape of connectors 38b is exemplified in FIGS. 6 and 7 as it is within the scope of the invention for the connectors 38b to have any shape.

In at least one embodiment, the closed pathways 12 are interconnected to form bands 34, as shown for example, in FIGS. 5-8 and 12-14. In some embodiments, the bands 34 extend longitudinally as indicated by bands 34b in FIG. 5. In other embodiments, the bands 34 extend circumferentially, as indicated by bands 34a in FIG. 5. The closed pathways 12 in adjacent bands 34 can be aligned, as shown for example in FIGS. 5, 7-8 and 12-14 or the closed pathways 12 in adjacent bands 34 can be offset, as shown for example in FIG. 6. In at least one embodiment, adjacent bands 34 define at least one opening 35, as shown for example in FIG. 6.

It is also within the scope of the invention for adjacent bands 34 of closed pathways 12 to be connected by any number of connectors 38, for example, one, two, three, four, five, six, seven, eight or more connectors 38. In some embodiments, flexibility is increased by limiting the number of connectors 38 between adjacent bands 34. Without being bound by theory, connectors engaging adjacent bands of closed pathways may allow for more flexibility along the stent length than adjacent bands that abut one another. It is within the scope of the invention for the connectors 38 engaging adjacent bands 34 to have any shape. For example, in FIG. 6, the connectors 38a have a Y-shaped structure while in FIG. 8, the connectors 38 are straight.

In at least one embodiment a plurality of closed pathways 12 are interconnected to form a complete stent pattern, as shown for example in FIG. 17. In some embodiments, the stent comprises a first section formed of a plurality of closed pathways having a first structure and a second section formed of a plurality of closed pathways having a second structure that is different from the first structure.

In some embodiments, at least two closed pathways 12 are interconnected to form at least one band 34. The at least one band 34 can be a longitudinal band that extends along at least a portion of the longitudinal length of the stent 10 and that extends about a portion of the circumference of the stent 10 as shown in FIG. 15, a circumferential band that extends about a portion of the circumference of the stent as shown in FIG. 16, and any combination thereof (not shown).

In at least one embodiment, the stent 12 has additional stent structure 32 in addition to the at least one closed pathway 12. In some embodiments, the additional structure comprises a plurality of serpentine bands of struts 22 that extend about at least a portion of the circumference of the stent. For example, FIG. 15 shows serpentine bands of struts 22 that extend about a portion of the circumference of the stent and FIG. 16 shows serpentine bands of struts 22 that extend about the entire circumference of the stent.

It is within the scope of the invention for a stent 10 to have one, two, three, four, five, six, seven or more bands 34 of closed pathways 12. It is also within the scope of the invention for a longitudinal band of closed pathways 12 to have any position about the circumference of the stent 10 and for a circumferential band of closed pathways 12 to have any position along the length of the stent 10. The circumferential bands of closed pathways 12 can be separated by additional stent structure 32, can abut one another, can be connected one to another by connectors 38, and any combination thereof. In at least one embodiment, a stent comprises a plurality of circumferential bands of closed pathways 12 wherein some of the circumferential bands have a plurality of closed pathways with a first structure and other of the circumferential bands have a plurality of closed pathways with a second structure different than the first structure.

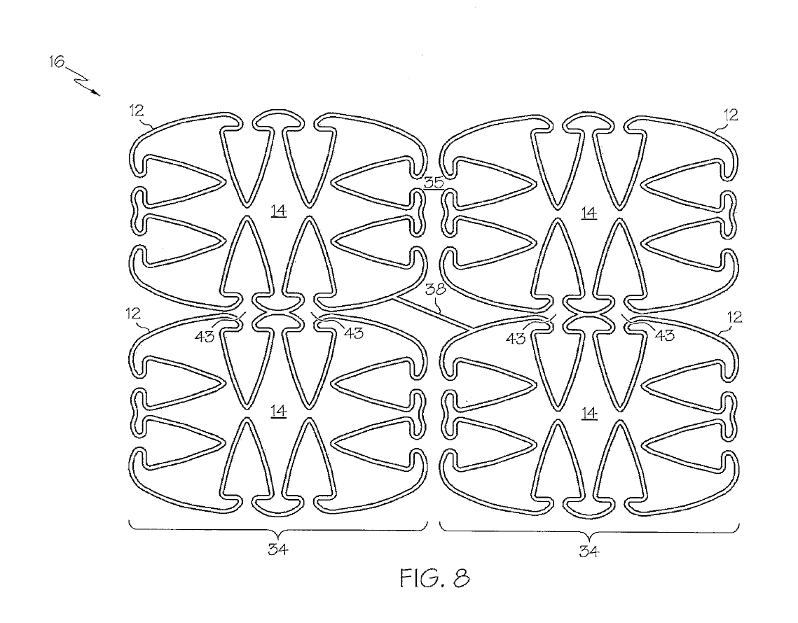

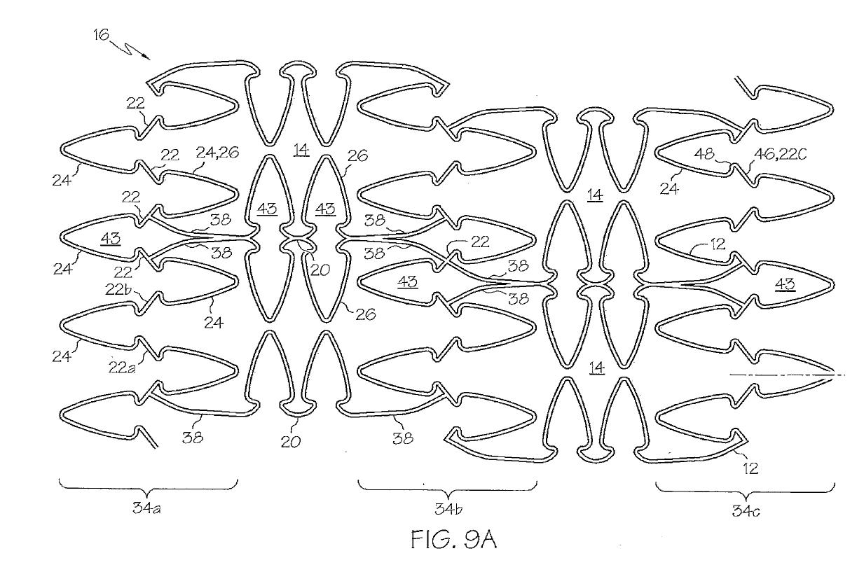

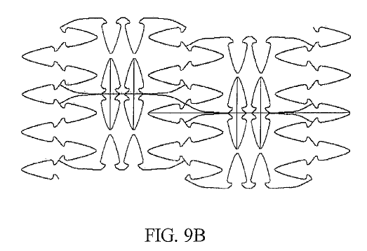

In some embodiments, each band 34 comprises a plurality of struts 22 interconnected by turns 24, as shown for example in FIGS. 9A and 9B. The patterns shown in FIGS. 9A and 9B the same except that the pattern in FIG. 9B has additional members 20b members 20b as discussed below in greater detail. In some embodiments, the turns 24 are petals 26. In some embodiments, each turn 24 extends from an end of a strut 22 to an end of a circumferentially adjacent strut 22. In other embodiments, each turn 24 is engaged to a turn 48 that is engaged to an end of a strut 22. In at least one embodiment, the orientation of the struts in a band 34 alternates from a first orientation and a second orientation where the second orientation is opposite to the first orientation, as shown for example by the struts 22 in band 34a of FIG. 9A. In some embodiments, the struts 22 are zig-zag struts. In one embodiment each zig-zag strut comprises two turns 48 engaged to a substantially straight middle portion 46 therebetween, as shown for example in FIG. 9A. In other embodiments, the struts 22 are substantially straight.

In at least one embodiment, each band 34 comprises a plurality of zig-zag struts 22 interconnected by turns 24 wherein the plurality of zig-zag struts 22 comprise first zig-zag struts 22a with a first orientation and second zig-zag struts 22b with a second orientation different than the first orientation wherein the first and second zig-zag struts 22a,b alternate along the length of the band 34. In at least one embodiment, circumferentially adjacent zig-zag struts are mirror images of one another. For example in FIG. 9A, strut 22a is a mirror image of strut 22b.

In one embodiment, each straight strut 22 is at an oblique angle relative to the longitudinal axis of the stent and the turns 48 are oriented in different directions. In some embodiments, the turns 48 are oriented in opposite directions relative to the longitudinal axis of the stent.

In at least one embodiment, the structure of the turn 24 is symmetrical about an axis that bisects the turn 24, as shown for example by axis A₁ of FIG. 9A. In some embodiments, the structure of the turn 24 is V-shaped. In other embodiments the structure of the turn 24 is U-shaped. In at least one embodiment, the axis bisecting each turn 24 is parallel to the longitudinal axis of the stent.

The pattern 16 shown in FIGS. 9A and 9B has at least one connector 38. In some embodiments, the connector 38 is engaged to at least one petal 26 and at least one strut 22. In some embodiments, the connector 38 is curvilinear, as shown for example in FIG. 9A. In other embodiments, the connector 38 is Y shaped and engages circumferentially adjacent circumferentially adjacent struts 22 that are engaged one to another by a turn 24. In at least one embodiment, a petal 26 engaged to the connector 38 is oriented in a circumferential direction.

In some embodiments, at least one connector 38 is engaged to a petal 26 oriented in a first circumferential direction and to another petal 26 oriented in a second circumferential direction opposite to the first circumferential direction, as shown for example in FIG. 9A. In some embodiments, the petals 26 are oriented on the same circumferential axis. In at least one embodiment, the petal 26, engaged to connector 38, is engaged to longitudinally adjacent petal 26 by member 20 to form a pair of petals. In some embodiments, the longitudinally adjacent petal 26 is engaged to another connector 38 that is engaged to another band 34. In at least one embodiment, longitudinally adjacent bands 34 are engaged one to another by at least two connectors 38 and at least one petal 26. In some embodiments, longitudinally adjacent bands 34 are engaged one to another two connectors 38, two petals 26, and one member 20.

In some embodiments, two connectors 38 are engaged to the same strut 22, as shown for example in FIG. 9A. In at least one embodiment, an end of the connector 38 is engaged approximately midway between the ends of the strut 22. In some embodiments, an end of the connector 38 is engaged to the middle portion 46 of a zig-zag strut 22. In some embodiments, a strut 22 engaged to at least one connector 38 is circumferentially adjacent to another strut 22 engaged to at least one connector 38. In one embodiment a strut 22 engaged to two connectors 38 is circumferentially adjacent to two struts 22 each engaged to only one connector 38.

FIG. 9B has members 20b in addition to the members 20 of FIG. 9A. As shown the members 20b are straight but it is within the scope of the invention for the members to have any shape. As shown in FIG. 9B, some of the members 20b are disposed within some of the openings 43. The member 20b bisects the opening 43 into two openings 43a,43b that are mirror images of one another. As shown in FIG. 9B, some of the openings 43 are bisected by a member 20b and some of the openings 43 are not bisected by a member 20b. In some embodiments, each opening 43 is bisected by a member 20b (not shown).

As shown in FIG. 9B, some of the members 20b are disposed within an opening defined by two interconnected petals 26. As shown, two members 20b are disposed within the opening defined by two interconnected petals 26. Each member 20b bisects the opening defined by the two interconnected petals 26 and the two members 20b are perpendicular to one another. Thus, thus there are four openings within the boundary of the two interconnected petals with each of the four openings being defined by a portion of each of the two members 20b and a portion of a petal 26. As shown in FIG. 9B, each of the four openings are mirror images of two of the other openings. Although two members 20b are shown within the opening 43 defined by two interconnected petals 26, it is within the scope of the invention for the opening 43 to have one member 20b disposed within opening 43.

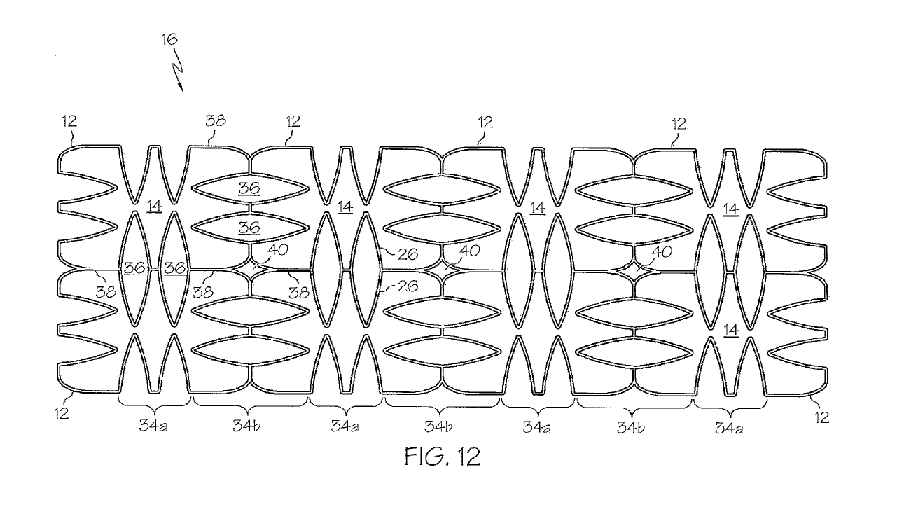

In at least one embodiment, a stent 10 has a plurality of diamond shaped openings 36, as shown, for example, in FIGS. 12-14. Each diamond shaped opening 36 has a length and a width, the length is greater than the width, with each of the length and width being measured from its greatest point. In at least one embodiment, each diamond shaped opening 36 is formed from two petals 26 engaged one to another at their wide ends. A diamond shaped opening 36 is oriented in a circumferential direction when its length is oriented circumferentially relative to the longitudinal axis of the stent and a diamond shaped opening 36 is oriented in a longitudinal direction when its length is oriented longitudinally relative to the longitudinal axis of the stent.

In some embodiments, the plurality of diamond shaped openings 36 comprises a first plurality of diamond shaped openings 36 oriented in a circumferential direction and a second plurality of diamond shaped openings 36 oriented in a longitudinal direction, as shown, for example, in FIGS. 12 and 13. In one embodiment, the first plurality of diamond shaped openings 36 is arranged in pairs and the second plurality of diamond shaped openings 36 is arranged in pairs.

In other embodiments, the stent 10 comprises a plurality of bands 34. In some embodiments the plurality of bands 34 extend circumferentially, as shown in FIG. 12 and in other embodiments the plurality of bands 34 extend longitudinal as shown in FIG. 13. The plurality of bands 34 comprises a plurality of first bands 34a comprising a plurality of diamond shaped openings 36 oriented in a circumferential direction and a plurality of second bands 34b comprising a plurality of diamond shaped openings 36 oriented in a longitudinal direction, as oriented in a longitudinal direction, as shown for example in FIGS. 12 and 13.

In one embodiment, the one of the first or second bands 34 further comprises a plurality of small diamond shaped openings 40. In at least one embodiment, the small diamond shaped opening 40 is positioned at the intersections of the first and second bands 34a,b. As shown in FIG. 12, the small diamond shaped openings 40 are positioned on an axis that bisects a first band 34a and on an axis that bisects a second band 34b. In at least one embodiment the small diamond shaped opening 40 has an area that is smaller than the area of a diamond shaped opening 36. In some embodiments, small diamond shaped opening 40 is at least 50% smaller than the diamond shaped opening 36.

As shown in FIGS. 12 and 13, the first and second bands alternate with one another. In one embodiment the diamond shaped openings 36 of the first band 34a are arranged in pairs and the diamond shaped opening 36 of the second band 34b are arranged in pairs. In some embodiments, the pairs of diamond shaped openings 36 in each first band 34a are aligned with one another. In other embodiments, the pairs of diamond shaped openings 36 in the second bands 34b are aligned with one another. In some embodiments, the diamond shaped openings 36 in each first band 34a are aligned on two parallel axes and the diamond shaped openings 36 in each second band 34b are aligned on two parallel axes. In other embodiments the second band 34b comprises an alternating pattern of a pair of diamond shaped openings 36 and a small diamond shaped opening 40 so that each pair of diamond shaped openings 36 is engaged to two small diamond shaped openings 40.

In some embodiments, circumferentially adjacent pairs of diamond shaped openings 36 in the first band 34a are not connected one to another but are connected to the second band 34b by at least one connector 38, as shown in FIG. 12. In some embodiments, the connector 38 is engaged to one of the plurality of small diamond shaped openings 42 of the second band 34b. In other embodiments, longitudinally adjacent pairs of diamond shaped openings 36 in the second band 34b are not connected one to another but are connected to the first band 34a by at least one connector 38, as shown in FIG. 13.

In at least one embodiment, the stent 10 comprises bands of diamond shaped openings 36 that are interconnected by a plurality of diamond shaped openings 36 that are oriented 90 degrees to the bands of diamond shaped openings 36, as shown in FIGS. 12 and 13.

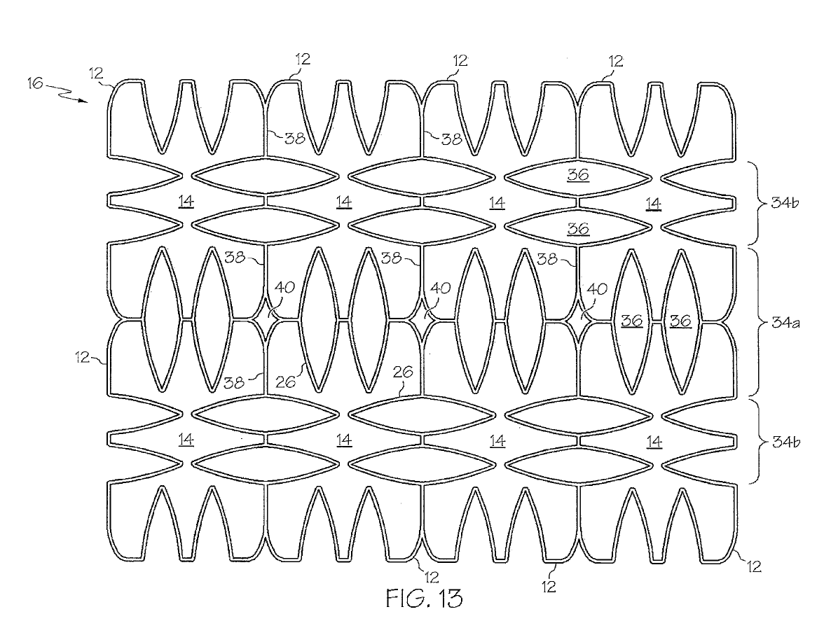

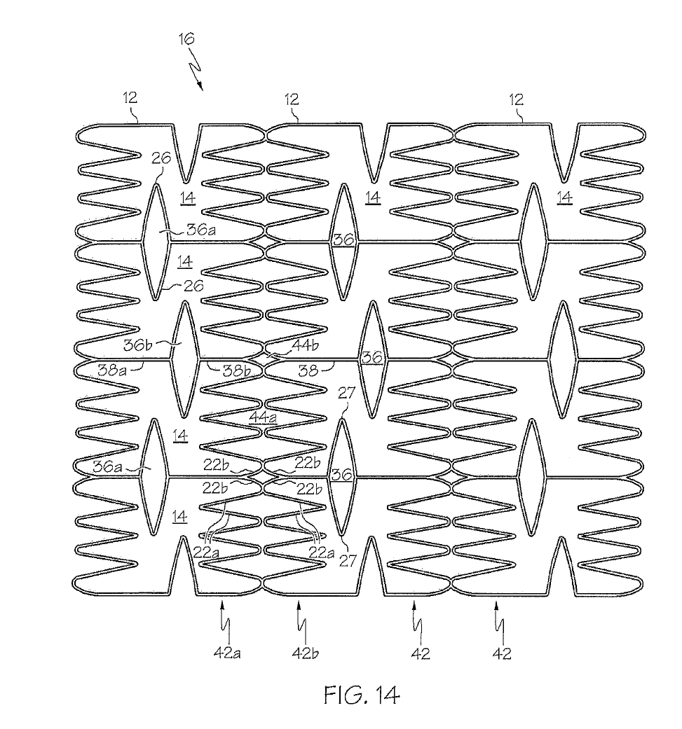

In some embodiments, each of the plurality of diamond shaped openings 36 are oriented in a circumferential direction, as shown for example in FIG. 14. In other embodiments, a stent 10 has a plurality of diamond shaped openings 36 and a plurality of serpentine bands 42 that extend about the circumference of the stent 10, as shown for example in FIG. 14. In some embodiments, the diamond shaped openings 36 are circumferentially oriented and circumferentially adjacent diamond shaped openings 36 are offset from one another. Thus, circumferentially adjacent diamond shaped openings 36 have different longitudinal positions. In some embodiments, the plurality of serpentine bands 42 comprises a plurality of first struts 22a and a plurality of second struts 22b, the second struts 22b having a shorter length than the first struts 22a. In one embodiment, the first and second struts 22a,b are arranged in a repeating pattern consisting of six first struts 22a and two second struts 22b (first-first-first-first-first-first-second-second).

In at least one embodiment, the stent 10 has a repeating pattern along the longitudinal axis that comprises a first serpentine band 42a, a second serpentine band 42b, the first serpentine band 32a engaged to a plurality of structures defining diamond shaped openings 36 and the second serpentine band 42b engaged to a plurality of structures defining diamond shaped openings 36, as shown, for example in FIG. 14. The first and second serpentine bands 42a,b each comprise a plurality of first struts 22a and a plurality of second struts 22b. In some embodiments, first struts 22a have a greater length than the second struts 22b. In some embodiments, the first serpentine band 42a abuts the second serpentine band 42b at the turns 24. In this embodiment the first and second serpentine bands 42a,b form a serpentine band pair. In one embodiment, the first and second serpentine bands 42a,b are out of phase. In some embodiments, the first and second struts 22a,b are arranged in a repeating pattern in each serpentine band 42 where the pattern is six first struts 22a followed by two second struts 22b.

In one embodiment, the pattern of the first serpentine band 42a is aligned with the pattern of the second serpentine band 42b so that the first struts 22a define first openings 44a and the second struts 22b define second openings 44b. In some embodiments, the second embodiments, the second openings 44b are smaller than the first openings 44a. In one embodiment, each first opening 44a is defined by six first struts 22a of the first serpentine band 42a and by six first struts 22a of the second serpentine band 42b and each second opening 44b is defined by two second struts 22b from the first serpentine band 42a and two second struts 22b from the second serpentine band 42b.

In at least one embodiment, a second opening 44b is engaged to two diamond shaped openings 36 by connectors 38 and abuts two first openings 44a. In some embodiments, the connectors 38 are straight. In other embodiments, the connectors 38 are longitudinally oriented or parallel to the longitudinal axis of the stent. In at least one embodiment, a plurality of diamond shaped openings 36 are positioned between two serpentine band pairs 42 where the plurality of diamond shaped openings 36 comprise at least two diamond shaped openings 36a with a first longitudinal position and at least two diamond shaped openings 36b with a second longitudinal position different than the first longitudinal position. In some embodiments, each diamond shaped opening 36 is engaged to a serpentine band 42 by a connector 38a and to a longitudinally adjacent serpentine band 42 by a connector 38b wherein the two connectors 38 are different lengths, as shown for example in FIG. 14.

In some embodiments, the stent 10 comprises at least one cell 14 and a plurality of openings each defined by a plurality of member 20 where the cell 14 is larger than each of the openings 35,36,40,43,44. In other embodiments, the stent 10 comprises at least one cell 14 with a geometry different than the geometry of other openings of the stent 10. This can be seen for example in FIGS. 5-9B and 12-14 where each cell 14 is larger than each of the other openings 35,36,40,43,44 and cell 14 has a different geometry than each of the other openings 35,36,40,43,44. In some embodiments, the size, the geometry, or a combination of size and geometry of the cell 14 provides for the proper positioning of a guide wire through the cell 14 defined by the closed pathway to be expanded into a side branch. This reduces incorrect placement of a guide wire so that portions of the stent that are not designed to be a side branch will not inadvertently be expanded outward when the stent is positioned within a body lumen, as discussed below in greater detail. In other embodiments, a greater number of shared members 20 between adjacent closed pathways 12 aids in the proper positioning of a guide wire through a cell 14 defined by a closed pathway 12.

In at least one embodiment, the placement of members 20b within openings 35,36,40,43,44 enhance the placement of a guide wire within cell 14. An example of members 20b within openings is shown for example in FIG. 9B, discussed above. As shown in FIGS. 9A and 9B, the placement of members 20b within openings further reduces the size of the opening relative to cell 14. This reduces incorrect placement of a guide wire so that portions of the stent that are not designed to be a side branch will not inadvertently be expanded outward when the stent is positioned within a body lumen, as discussed below in greater detail. Although not shown, it is within the scope of the invention for the openings of the other stent patterns shown to have members placed within to further reduce the size of the opening relative to cell 14. Moreover, although the members 20b shown in FIG. 9B bisect the openings 43, it is within the scope of the invention for member 20b not to bisect the opening so long as that the size of the opening relative to cell 14 is further reduced and the petals can extend away from the tubular wall of the stent when the closed pathway is expanded to form a side branch (not shown).

In at least one embodiment, the stent 10 includes at least one area, band, coating, member, detectable by imaging modalities such as X-Ray, MRI, ultrasound, etc. As shown in FIG. 28, radiopaque markers 30 can be used to position a closed pathway 12 relative to an ostium. It is within the scope of the invention for the radiopaque markers 30 to have any shape and any size. It is also within the scope of the invention for a stent to have radiopaque markers that are the same shape, same size, different shapes, different sizes, and any combination thereof. In at least one embodiment, the radiopaque makers 30 are positioned between bands 34 of closed pathways 12, as shown for example in FIG. 17. In some embodiments, the radiopaque markers 30 are positioned on either side of the closed pathway 12 to indicate the edges of the closed pathway 12. In at least one embodiment, the radiopaque markers 30 are positioned on an axis that is parallel to the longitudinal axis of the stent, as shown, for example in FIG. 17.

In at least one embodiment, a portion of at least one member 20 of a closed pathway 12 is radiopaque. In some embodiments, an entire petal 26 is radiopaque. In other embodiments, the second/tip end region 27 of at least one petal 26 of a closed pathway 12 is radiopaque. In at least one embodiment, at least one circumferentially oriented petal of a circumferentially oriented petal of a closed pathway does not define an opening (not shown). Thus, for example, the entire petal is arrow shaped. In this embodiment, the solid petal is a radiopaque marker.

In some embodiments the delivery system or other portion of the assembly may include one or more areas, bands, coatings, members, etc. that is (are) detectable by imaging modalities such as X-Ray, MRI, ultrasound, etc. In some embodiments at least a portion of delivery system or assembly is at least partially radiopaque.

FIG. 18 shows the stent pattern of FIG. 5 in a reduced state. As shown, the circumferentially aligned petals 26 overlap one another when the stent is in the reduced state. In at least one embodiment, a stent design comprising circumferentially petals that overlap provides circumferential support while maintaining a low profile for delivery. Note that the offset peaks of the closed pathway 12 shown in FIG. 11 do no overlap when the stent is in a reduced state.

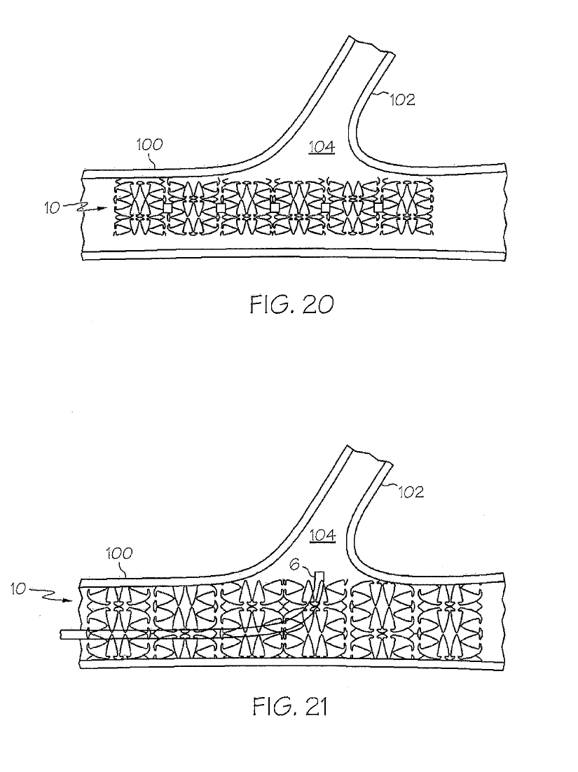

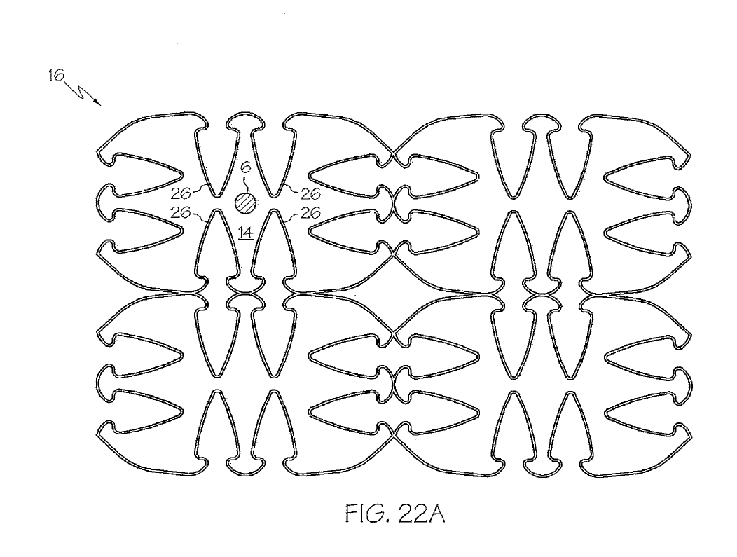

In at least one embodiment, a closed pathway is expanded to form a side branch, as shown in FIGS. 20-24 which are schematic illustrations of a stent comprising a plurality of closed pathways being delivered to a body lumen with a side branch lumen extending therefrom. FIG. 20 shows the stent 10 in an unexpanded reduced state. When the stent 10 is in a reduced state, some of the petals 26 of the closed pathway(s) of the stent 10 overlap other of the petals 26 of the same closed pathway, as discussed below in greater detail. FIG. 21 shows the stent 10 in an expanded state with a guide wire 6 extending through one of the plurality of closed pathways 12. When the stent 10 is in the expanded state, the petals 26, which were overlapping when the stent 10 was in the reduced state, no longer overlap one another.

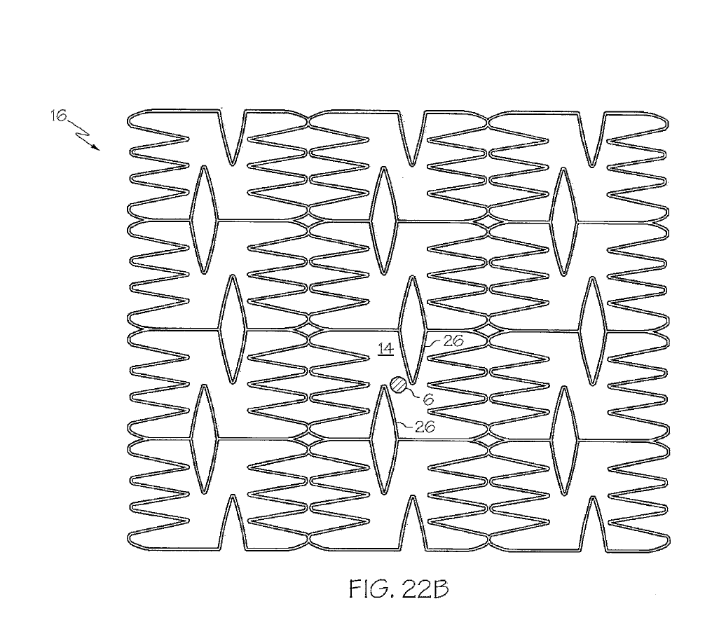

For simplicity, the flat views of FIGS. 22a and 22b show the position of the guide wire 6 within a cell 14 defined by one of the plurality of closed pathway in a pattern. FIG. 22a shows the pattern 16 of FIG. 5 with a guide wire 6 extending through a cell 14 defined by one of the closed pathways 12. As shown in FIG. 22a, the guide wire 6 extends through the cell 14 in the area of the cell 14 that is surrounded by the circumferentially oriented petals 26. FIG. 22b shows the pattern 16 of FIG. 14 with a guide wire 6 extending through a cell 14 defined by one of the closed pathways 12. In this geometry, the guide wire 6 extends between the circumferential petals 26 that are longitudinally offset from one longitudinally offset from one another. As discussed above, the size, the geometry, combination of size and geometry of the cell 14 compared to the other openings of the stent 10 allows for the proper positioning of the guide wire 6 within the cell 14.

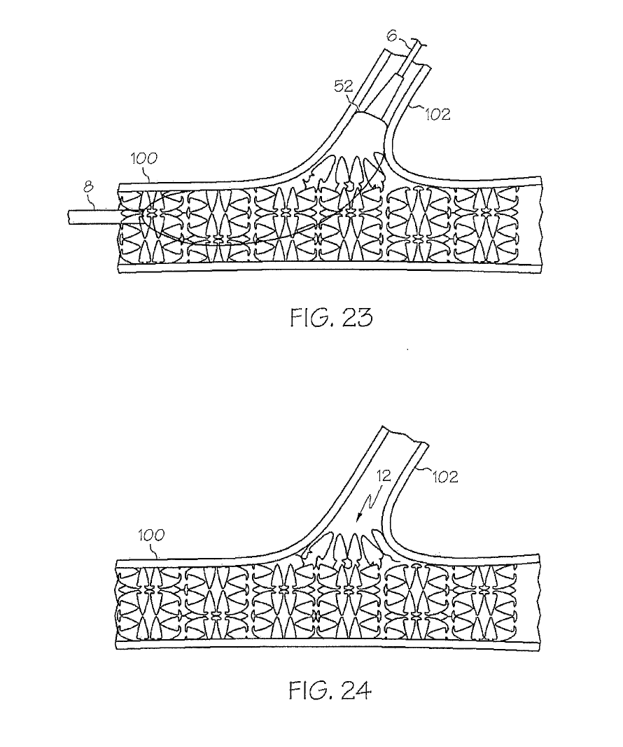

FIG. 23 shows one of the closed pathway 12 of the stent 10 being expanded by a balloon catheter 8 extending through the closed pathway 12 to form a side branch that extends into the side branch vessel 102. As shown, the balloon catheter 8 is disposed over the guide wire 6, as is known in the art.

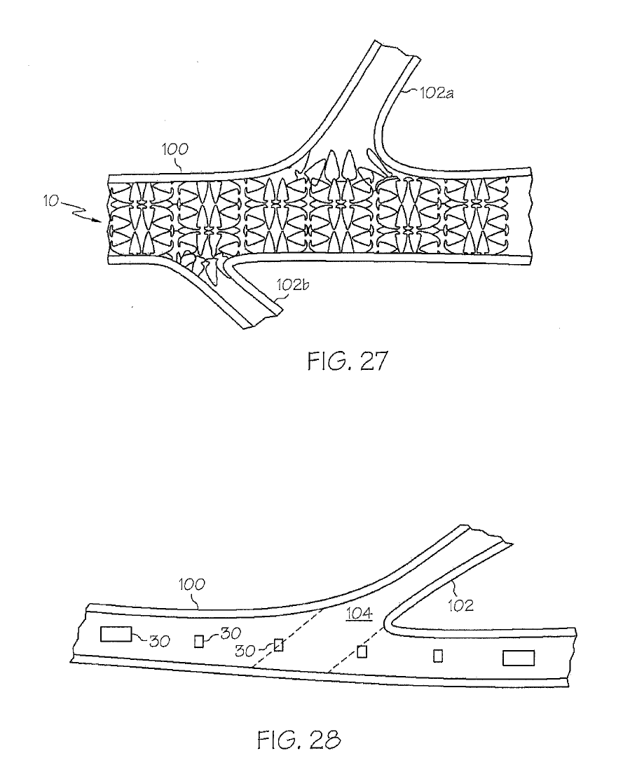

In at least one embodiment, a plurality of closed pathways 12 of the stent 10 is expanded with each expanded closed pathway 12 each forming a side branch extending into a side branch vessel 102, as shown for example in FIG. 27. In some embodiments, the ostial support provided the a closed pathway 12 forming a side branch allows side branch stents to be overlapped in provisional T procedures in which a second stent is deployed in the side branch vessel.

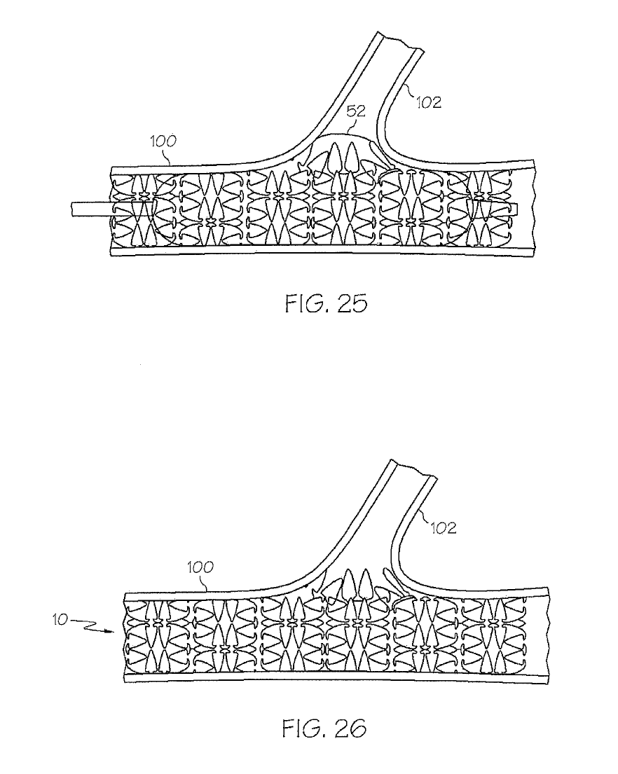

In at least one embodiment, a closed pathway 12 is at least partially expanded to form a side branch by the same balloon catheter 8 that expands the stent 10, as shown for example in FIGS. 25-26. In one embodiment, the balloon 52 of the balloon catheter 8 is made of compliant material. In this embodiment, due to the ostium of the side branch, the expansion of a closed pathway 12 into the side branch is initiated by the balloon 52. In some embodiments, a second balloon catheter 8 is used to further expand the partially expanded closed pathway 12.

In at least one embodiment, the deployment of the closed pathway 12 to form a side branch is not dependent on the angle of the side branch vessel 102 relative to the main branch vessel 100. Thus, a closed pathway 12 can be deployed as a side branch in a side branch vessel 102 that has any angle relative to the main vessel 100.

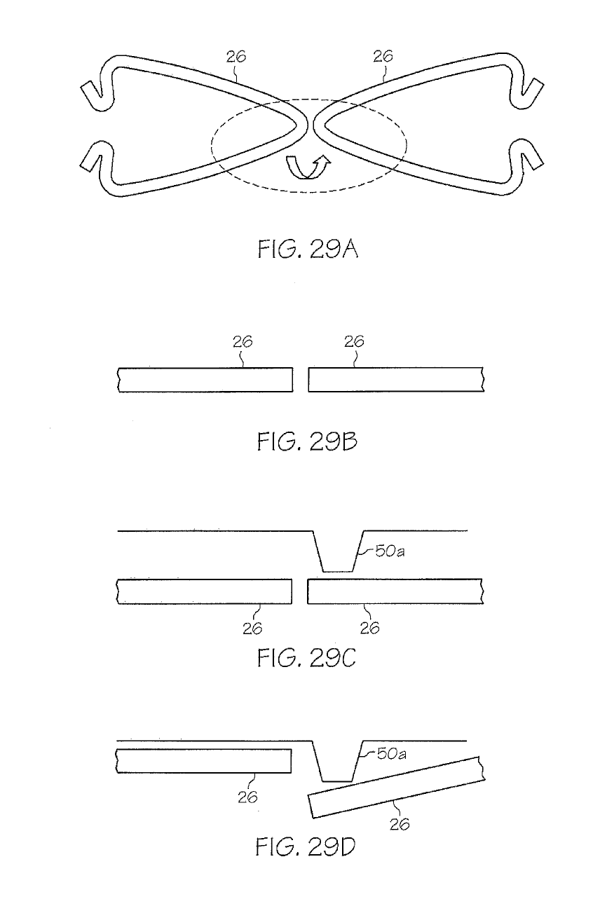

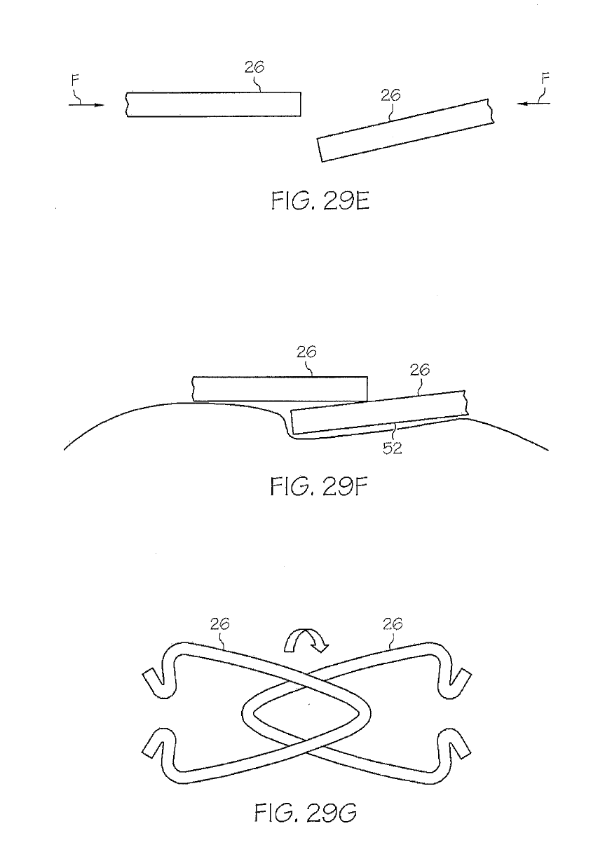

As shown in FIG. 18, in some embodiments adjacent petals 26 oriented in a circumferential direction overlap one another when the stent 10 is in a reduced state. As shown, the second/tip end region 27 of one petal overlaps the second/tip end region 27 of the other petal. FIGS. 29A-29G is a schematic of a method of crimping a balloon expandable stent into a reduced state in which adjacent circumferentially oriented petals 26 overlap one another. Note that if the stent is a self-expanding stent, the stent is programmed to assume the reduced state, as is known in the art. FIG. 29A is a top view of two adjacent petals 26 in a pre-reduced state. When the stent is in the pre-reduced state, none of the petals 26 overlap state, none of the petals 26 overlap one another. FIG. 29B is a side view of the two adjacent petals 26 of FIG. 29A. Note that the two petals 26 are facing opposite directions so that they are oriented 180 degrees from one another.

FIG. 29C shows a step in the crimping process wherein a first crimping member 50a is aligned with one of the adjacent petals 26. FIG. 29D shows a pre-reduction step wherein one of the adjacent petals 26 is angled relative to the other petal 26. In at least one embodiment, each petal 26 facing a first direction is angled relative to another petal 26 angled in a second direction wherein the first direction is 180 degrees to the second direction.

After the petals 26 are angled, the stent is reduced in diameter by second crimping members (not shown). It is within the scope of the invention for the first and second crimping members to have any shape. An example of a shape for a crimping member is shown in FIG. 29C. During the reduction or crimping of the stent, a portion of the petal 26 which was not angled during the pre-reduction step moves over a portion of the petal 26 which was angled during the pre-reduction step, as shown in FIGS. 29E and 29F. As shown in FIG. 29F, after the stent is reduced, the petal 26 which was angled during the pre-reduction step is adjacent to the balloon 52 and the petal 26 which was not angled during the pre-reduction step has a portion which is not adjacent to the balloon 52. FIG. 29G is a top view of FIG. 29F. Once the stent is reduced, it is ready to be delivered to a body lumen, as discussed above in reference to FIGS. 20-24 and 25-26.

In at least one embodiment, adjacent petals that overlap one another in a reduced state have second/tip end regions 27 that are angled or rounded (not shown). In this embodiment, the second/tip end region 27 of one petal is angled or rounded in a first direction and the second/tip end 27 of a second petal is angled or rounded in a second direction opposite of the first direction. Without being bound by theory, angling/rounding the second/tip end region 27 of the petals in opposite directions allows the petals to slide over one another. In one embodiment, petals with second/tip end region 27 that are angled or rounded relative to one another do not need to undergo the pre-reduction step of the crimping process discussed above.

In other embodiments, circumferentially adjacent petals that extend in a longitudinal direction overlap one another (not shown). In one embodiment, petals having a wide base are reduced so that circumferentially adjacent petals that are longitudinally oriented longitudinally oriented overlap one another. This aids in reducing the diameter of the stent for delivery.

In some embodiments, the first and second crimping members 50 form a part of a single crimping device. In other embodiments the first crimping members 50a form a part of a first crimping device and the second crimping members form a part of a second crimping device. A non-limiting example of a crimping device that can be used to crimp a stent to its reduced state as discussed above, is shown in commonly assigned U.S. Application Publication No. 2007/0123970. Using this crimping device, one set of crimping members modified the position of a first set of petal peaks and then the other set of crimping members overlaps a second set of petal peaks over the first set of petal peaks, as discussed above.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A stent, the stent comprising at least one closed pathway, the at least one closed pathway defining a single cell and comprising a plurality of members, the plurality of members comprising:
a plurality of first members, each first member having a first shape;
a plurality of second members, each second member having a second shape different than the first shape; and
a plurality of petals, the plurality of petals comprising a plurality of first petals and a plurality of second petals, each first petal being bisected by a first axis and each second petal being bisected by a second axis, the first axis being perpendicular to the second axis;
wherein one of the first axes intersecting at least one of the second petals.

2. The stent of claim 1, the plurality of members comprising four first members, four second members and eight petals.

3. The stent of claim 1, each first member engaging two petals where one petal is bisected by a first axis and the other petal is bisected by a second axis, each second member engaging either two petals each being bisected by a first axis or two petals each being bisected by a second axis.

4. The stent of claim 1, wherein the cell is symmetrical about at least one axis.

5. The stent of claim 1, the stent having a reduced state, wherein two petals on one of a first axis and a second axis overlap one another.

6. The stent of claim 1, each second member being positioned opposite another second member.

7. The stent of claim 2, the members of each closed pathway being arranged as follows:
first member-petal-second member-petal-first member-petal-second member-petal-first member-petal-second member-petal-first member-petal-second member-petal.

8. The stent of claim 1, the plurality of members further comprising a plurality of third members having a third shape, the third shape being different than the first and second shapes.

9. The stent of claim 8, the plurality of members of each closed pathway being arranged as follows: first member-petal-second member-petal-first member-petal-third member-petal-first member-petal-second member-petal-first member-petal-third member-petal.

10. The stent of claim 8, the third member being positioned opposite of another third member.

11. The stent of claim 1, the at least one closed pathway comprising a plurality of closed pathways, each pathway having the same arrangement of the plurality of members.

12. The stent of claim 11, the plurality of closed pathways arranged in at least one band that extends around a circumference of the stent.

13. The stent of claim 11, the plurality of closed pathways arranged in at least one band that extends along a longitudinal length of the stent.

14. The stent of claim 11, the plurality of closed pathways being connected one to another by at least one connector.

15. The stent of claim 11, the plurality of closed pathways abutting one another.
